(19) United States
(12) Patent Application Publication
(10) Pub. No.: US 2023/0372021 A1
(43) Pub. Date: Nov. 23, 2023

(54) DISPLAYING ORTHOGRAPHIC AND ENDOSCOPIC VIEWS OF A PLANE SELECTED IN A THREE-DIMENSIONAL ANATOMICAL IMAGE

(71) Applicant: BIOSENSE WEBSTER (ISRAEL) LTD., Yokneam (IL)

(72) Inventors: Fady Massarwa, Baka Al Gharbiyya (IL); Sigal Altman, Ramat Hashofet (IL); Amir Ben-Dor, Ramat Hashofet (IL); Gal Bar Zohar, Yokneam Moshava (IL)

(21) Appl. No.: 17/749,654

(22) Filed: May 20, 2022

(51) Int. Cl. A61B 34/20; G06T 19/20; A61B 18/14; A61B 90/00

(52) U.S. Cl. CPC A61B 34/20; G06T 19/20; A61B 18/1492; A61B 90/37; G06T 2219/2016; G06T 2210/41; A61B 2018/00351

(57) ABSTRACT

A method includes inserting a catheter into an organ of a patient and selecting, in a three-dimensional (3D) image of the organ, a plane of interest (POI). A first image, which includes an endoscopic view of the 3D image from a direction facing the POI, is produced. A second image, which includes a sectional view of the 3D image that is clipped by the POI, is produced, and the first and second images are displayed to a user.

### FIELD OF THE DISCLOSURE

The present disclosure relates generally to graphical user interface, and particularly to methods and system for displaying orthographic and endoscopic views of a common plane selected in a three-dimensional (3D) anatomical image.

### BACKGROUND OF THE DISCLOSURE

Various techniques for visualizing organs in multiple perspectives and using of virtual imaging have been published.

For example, U.S. Pat. No. 7,477,768 describes methods for generating a three-dimensional visualization image of an object, such as an internal organ, using volume visualization techniques. The techniques include a multi-scan imaging method; a multi-resolution imaging method; and a method for generating a skeleton of a complex three-dimension object. The applications include virtual cystoscopy, virtual laryngoscopy, virtual angiography, among others.

### BRIEF DESCRIPTION OF THE DRAWINGS

The present disclosure will be more fully understood from the following detailed description of the examples thereof, taken together with the drawings in which:

FIG. 1 is a schematic, pictorial illustration of a catheter-based position-tracking and ablation system, in accordance with an example of the present disclosure;

FIG. 2 is a schematic, pictorial illustration of a three-dimensional (3D) visualization of a heart using a combination of exterior and endoscopic views, in accordance with an example of the present disclosure;

FIG. 3A is a schematic, pictorial illustration of a clip plane (CP) of a selected plane of interest (POI) in heart, in accordance with an example of the present disclosure;

FIG. 3B is a schematic, pictorial illustration of selected plane of interest (POI) in heart, in accordance with an example of the present disclosure;

FIG. 3C is a schematic, pictorial illustration of a sectional view of a POI selected in heart, in accordance with an example of the present disclosure;

FIG. 4 is a schematic, pictorial illustration of a 3D visualization of pulmonary veins (PVs) using a combination of a sectional view clipped by a selected plane of interest (POI) in a 3D image, and an endoscopic view from a direction facing the selected POI, in accordance with an example of the present disclosure; and

FIG. 5 is a flow chart that schematically illustrates a method for visualizing a heart using a combination of a sectional view clipped by a selected POI of a 3D image and an endoscopic view from a direction facing the selected POI, in accordance with an example of the present disclosure.

### DETAILED DESCRIPTION OF EXAMPLES

### Overview

Examples of the present disclosure that are described hereinbelow provide improved techniques for displaying to a user of a catheter-based position-tracking and ablation system, a combination of different views of at least a section in an organ of a patient.

In the present example, the organ comprises a heart, which is intended to be ablated using any suitable technique, such as radiofrequency (RF) ablation, for treating arrhythmia in the patient heart. In RF ablation, a user of the system, e.g., a physician, inserts a catheter into the heart, and based on an Electrophysiology (EP) mapping of the heart, the user selects, in heart tissue, one or more sites intended to receive RF ablation signals. In response to applying the ablation signal, cells of the tissue in the ablated sites are killed and being transformed into lesions that cannot conduct electrophysiological signals. Because tissue ablation is an aggressive and typically irreversible, it is important to apply the RF signals to the tissue, very accurately at the selected sites. Thus, the physician typically needs a combination of: (i) a general view of the heart, and (ii) a high-resolution image of the site(s) intended to be ablated.

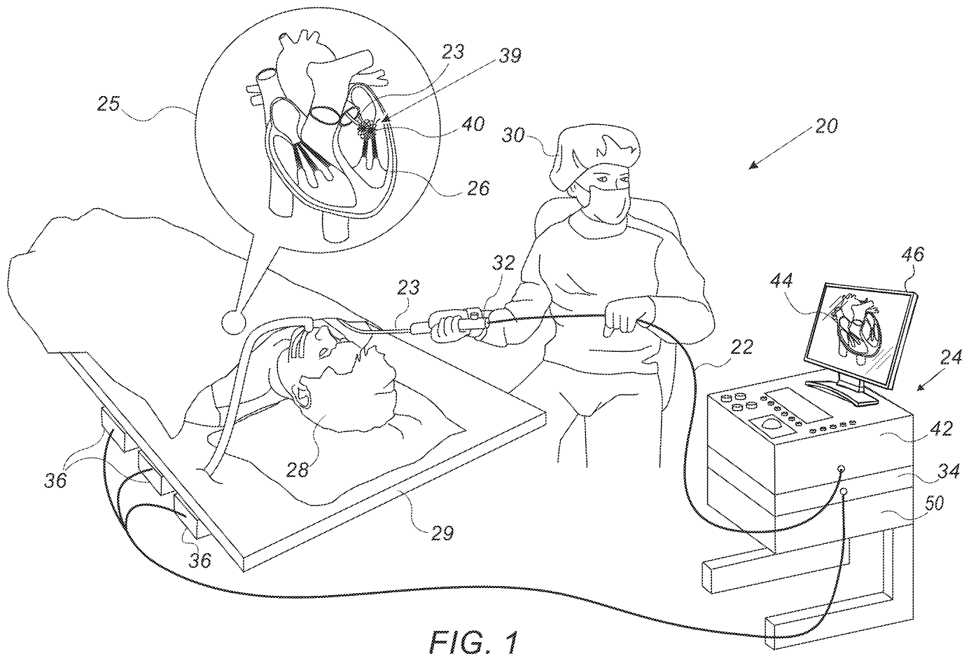

In some examples, a catheter-based position-tracking and ablation system comprises a catheter configured for performing the ablation, a processor, and a display. The catheter may comprise one or more position sensors of a position tracking system described in FIG. 1 below. The position sensors are configured to produce position signals indicative of the position of the catheter in the heart.

In some examples, the processor is configured to receive an anatomical image of the heart and the position signals, and to display the position of a distal-end assembly (DEA) of the catheter overlaid on a three-dimensional map of the heart. The physician can navigate the DEA to the ablation sites, and subsequently, apply the RF signals to the tissue at the selected ablation sites.

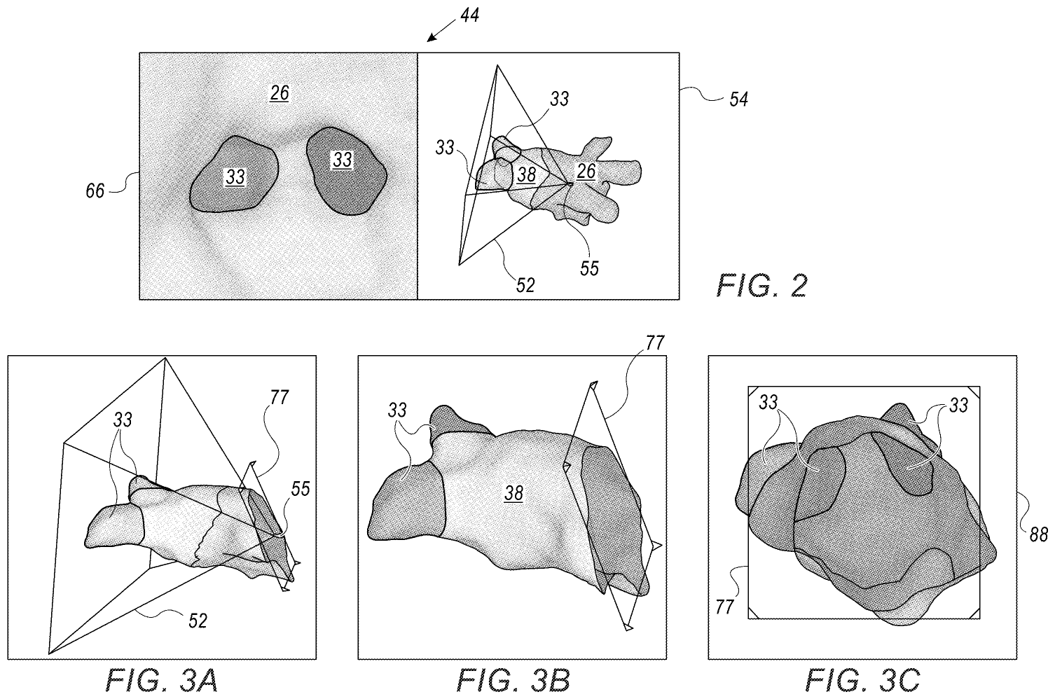

In some examples, the processor is configured to display to the physician various types of images. For example, a three-dimensional (3D) image of an exterior view of the heart, together with an endoscopic view of a site or a section intended to be ablated. Note that the endoscopic view may be produced using a virtual camera described in detail in FIG. 2 below.

The endoscopic view provides the physician with anatomical details of a section of the heart intended to be ablated. The anatomical details are important for planning the ablation but may be insufficient because the endoscopic view provides the physician with a perspective image, so that anatomical elements located in close proximity to the virtual camera appear larger than other anatomical elements located farther from the virtual camera. Moreover, when looking at the endoscopic view, the physician may lose sense of orientation in the image within the heart, because he/she may not observe features (e.g., veins) in the anatomy in the way the features appear in the exterior view, and such features improve the sense of orientation. Thus, separate images of the exterior view and the endoscopic view may provide the physician with sufficient imaging for performing the ablation in optimal conditions

In other examples, the processor is configured to produce: (i) a sectional view clipped by a plane of interest (POI) selected in a 3D image of the heart, and (ii) an endoscopic view from a direction facing the selected POI. In some examples, the processor is configured to produce a clipping plane, also referred to herein as a clip plane, of the POI, and to rotate the image of the POI so that the orientation of the sectional view of the POI, and the endoscopic view are similar. Note that the sectional view is not affected by the topography of the section in question of the heart, and only presents a graphical representation of the topography. In other words, the sectional view ignores the topography, and therefore, provides the physician with a complementary view of the section in question.

In some examples, the processor is configured to produce the endoscopic image by: (i) positioning, within the 3D image of the heart, the virtual camera at a given position and a given orientation, and (ii) defining one or more imaging parameters, such as but not limited to magnification and/or one or more angle(s) of view.

In some examples, the processor is configured to select the given position and the given orientation of the virtual camera for displaying a section of the heart, which comprises one or more of the ablation sites.

In some examples, the processor is configured to produce the image of the sectional view of the POI by producing a graphic representation of the clip plane of the POI, and subsequently, displaying on the display the sectional view of the clip plane. In such examples, the processor is configured to present the field-of-view (FOV) of both: (i) the endoscopic image, and (ii) the sectional view of the clip plane of the POI, in an orientation parallel to the plane of the system display.

In some examples, the FOVs may be presented side-by-side on the system display. In such examples, the physician can see both FOVs at the same time.

In other examples, the processor is configured to display on the display the two FOVs, also referred to herein as first and second images, by toggling between the display of the first and second images. In one implementation, the processor is configured to: (i) display the first image when applying to the system display a first range of zoom values, and (ii) display the second image when applying to the display a second range of zoom values, different from the first range of zoom values. In another implementation, the processor is configured to display the 3D image of the exterior view of the heart, instead of the first image or instead of the second image. In this implementation, the processor may apply to the display a third range of zoom values, different from the first range of zoom values and the second range of zoom values, thereby, the physician may toggle between the three images using the zoom-in and zoom-out functions of the processor on the system display.

In alternative examples, the processor may customize the presentation of the three images described above using any other suitable configuration.

The disclosed techniques improve the visualization of organs (e.g., heart) of a patient during tissue ablation and other sorts of minimally invasive medical procedures. Such procedures typically require both (i) general view image(s) that are not distorted by the topography of the organ in question, and (ii) high-resolution images of the same plane of interest in the organ in question. This combination provides the physician with improved imaging that helps to improve the quality of tissue ablation and other sorts of medical procedures.

### System Description

FIG. 1 is a schematic, pictorial illustration of a catheter-based position-tracking and ablation system 20, in accordance with an example of the present disclosure. In some examples, system 20 comprises a catheter 22, in the present example an expandable cardiac catheter described below, and a control console 24. In the example described herein, catheter 22 may be used for any suitable therapeutic and/or diagnostic purposes, such as but not limited to sensing of electro-anatomical (EA) information in tissue in question and applying ablation signals to tissue of a heart 26 (inset 25). In the context of the present disclosure, the term information refers to the spatial location of the catheter distal end, and an electrocardiogram (ECG) signal sensed by electrodes of catheter 22.

In some examples, console 24 comprises a processor 42, typically a general-purpose computer, with suitable front end and interface circuits for receiving signals from catheter 22 and for controlling other components of system 20 described herein. Processor 42 may be programmed in software to carry out the functions that are used by the system, and is configured to store data for the software in a memory 50. The software may be downloaded to console 24 in electronic form, over a network, for example, or it may be provided on non-transitory tangible media, such as optical, magnetic, or electronic memory media. Alternatively, some or all of the functions of processor 42 may be carried out using an application-specific integrated circuit (ASIC) or any suitable type of programmable digital hardware components.

Reference is now made to an inset 25. In some examples, catheter 22 comprises a distal-end assembly (DEA) 40 having an expandable member (e.g., a balloon or a basket), and a shaft 23 for inserting DEA 40 to a target location for ablating tissue in heart 26. During an Electrophysiology (EP) mapping and/or ablation procedure, physician 30 inserts catheter 22 through the vasculature system of a patient 28 lying on a table 29. Physician 30 moves DEA 40 to the target location in heart 26 using a manipulator 32 near a proximal end of catheter 22, which is connected to interface circuitry of processor 42. In the present example, the target location may comprise tissue having one or more sites intended to be ablated by DEA 40.

In some examples, catheter 22 comprises a position sensor 39 of a position tracking system, which is coupled to the distal end of catheter 22, e.g., in close proximity to DEA 40. In the present example, position sensor 39 comprises a magnetic position sensor, but in other examples, any other suitable type of position sensor (e.g., other than magnetic based) may be used.

Reference is now made back to the general view of FIG. 1. In some examples, during the navigation of DEA 40 in heart 26, processor 42 receives signals from magnetic position sensor 39 in response to magnetic fields from external field generators 36, for example, for the purpose of measuring the position of DEA 40 in heart 26. In some examples, console 24 comprises a driver circuit 34, configured to drive magnetic field generators 36. Magnetic field generators 36 are placed at known positions external to patient 28, e.g., below table 29.

In some examples, processor 42 is configured to display, e.g., on a display 46 of console 24, the tracked position of DEA 40 overlaid on an image 44 of heart 26, which is typically a three-dimensional (3D) image.

The method of position sensing using external magnetic fields is implemented in various medical applications, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publication Nos. 2002/0065455 A1, 2003/0120150 A1 and 2004/0068178 A1.

### Displaying a Combination of Exterior View of Patient Heart and Endoscopic View of a Section in Heart

FIG. 2 is a schematic, pictorial illustration of (i) an exterior view 54, and (ii) an endoscopic view 66, presented side-by-side in 3D image 44 of heart 26, in accordance with an example of the present disclosure.

Reference is now made to a 3D image of exterior view 54 of heart 26. In some examples, processor 42 is configured to produce a virtual camera at a given position and a given orientation within exterior view 54 of heart 26.

In some examples, processor 42 is configured to define a 3D field-of-view (FOV) 52 (shown as a virtual pyramid) of virtual camera 55. Processor 42 is configured to define 3D FOV 52 by determining imaging parameters of virtual camera 55. For example, one or more angles of view (e.g., three angles of view) define the direction and an opening angle of the pyramid, and a magnification may define a 3D section 38, which is imaged by virtual camera 55, and a magnification of endoscopic view 66 of the anatomical features within 3D FOV 52. Note that the virtual images produced by virtual camera 55 are based on any suitable pre-acquired anatomical images and/or anatomical mapping information stored in memory 50 and/or in processor 42. For example, processor 42 may receive one or both of: (i) computerized tomography (CT) images acquired by a CT imaging system, and (ii) fast anatomical mapping (FAM) of heart 26 produced by moving a catheter at registered positions within cavities of heart 26.

In the present example, 3D FOV 52 acquires section 38 of heart 26 having, inter alia, two pulmonary veins (PVs) 33. In this example, the ablation procedure comprises a PV isolation of one or both of PVs 33, and DEA comprises a balloon having ablation electrodes disposed on an expandable member of the balloon. During the PV isolation procedure, physician 30 inserts the balloon into the ostium of a selected PV 33, and subsequently, inflates the balloon to place the ablation electrodes in contact with the tissue intended to be ablated. After verifying sufficient contact force between the ablation electrodes and the tissue, physician 30 may use system 20 to apply radiofrequency (RF) signals to the electrodes for ablating the tissue.

Reference is now made to endoscopic view 66. In some examples, endoscopic view 66, also referred to herein as a “first image” provides physician 30 with anatomical details of PVs 33 and the tissue surrounding PVs 33. The details are important for planning the ablation but may be insufficient because endoscopic view 66 is a perspective image, so that anatomical elements located in close proximity to virtual camera 55 appear larger than other anatomical elements located farther from virtual camera 55. Moreover, when looking at endoscopic view 66, physician 30 may lose sense of orientation in the image because, because he/she may not observe endoscopic view 66 features in the anatomy in the way the features appear in the exterior view. For example, veins and other anatomical features may help the physician in improving the sense of orientation while performing the procedure within heart 26. Thus, a combination of exterior view 54 and endoscopic view 66 may not provide physician 30 with sufficient optimal imaging for performing the ablation of one or more PVs 33.

FIG. 3A is a schematic, pictorial illustration of a clip plane (CP) of a selected plane of interest (POI) 77 shown in exterior view 54 of heart 26, in accordance with an example of the present disclosure.

In some examples, processor 42 is configured to select POI 77 based on: (i) the position of virtual camera within the 3D image of exterior view 54 of heart 26 (shown in FIG. 2 above), and (ii) the orientation of the virtual camera, and optionally, imaging parameters of endoscopic view 66 of FIG. 2 above.

In some examples, in FIG. 3A processor 42 defines POI 77 within the volume of the full map of heart 26, and produces a clip plane (CP) of POI 77. Note that the section of heart 26 that is not “imaged” by virtual camera 55 is removed from the image, and POI 77 is positioned at the edge of the map shown in FIG. 3A.

FIG. 3B is a schematic, pictorial illustration of plane of interest (POI) 77, which is selected by processor 42 to define a section of exterior view 54 of heart 26, in accordance with an example of the present disclosure.

In some examples, processor 42 is configured to produce POI 77 that provides physician 30 with: (i) a graphic representation of the clip plane of POI 77, and (ii) presents on the display the sectional view of the clip plane of POI 77. Note that in the example of FIG. 3B, the presented orientation of POI 77 is not parallel to the orientation of endoscopic view 66 of FIG. 2 above, and therefore, physician 30 cannot see the anatomical elements of the sectional view of the clip plane of POI 77.

FIG. 3C is a schematic, pictorial illustration of an image of a sectional view 88 of POI 77, in accordance with an example of the present disclosure.

In some examples, processor 42 is configured to produce sectional view 88 by rotating the image shown in FIG. 3B, such that POI 77 is parallel to display 46 of console 24 (shown in FIG. 1 above).

In some examples, the image of sectional view 88 comprises a sectional view of the 3D image of the selected section of heart 26 (shown in FIGS. 3A and 3B above) clipped by POI 77. In some examples, an image of sectional view 88 comprises the sectional view of PVs 33 and tissue of the surrounding wall of the respective cavity (e.g., an atrium) of heart 26.

### Displaying Endoscopic View Together with Sectional View of 3D Image Clipped by a Selected Plane of Interest

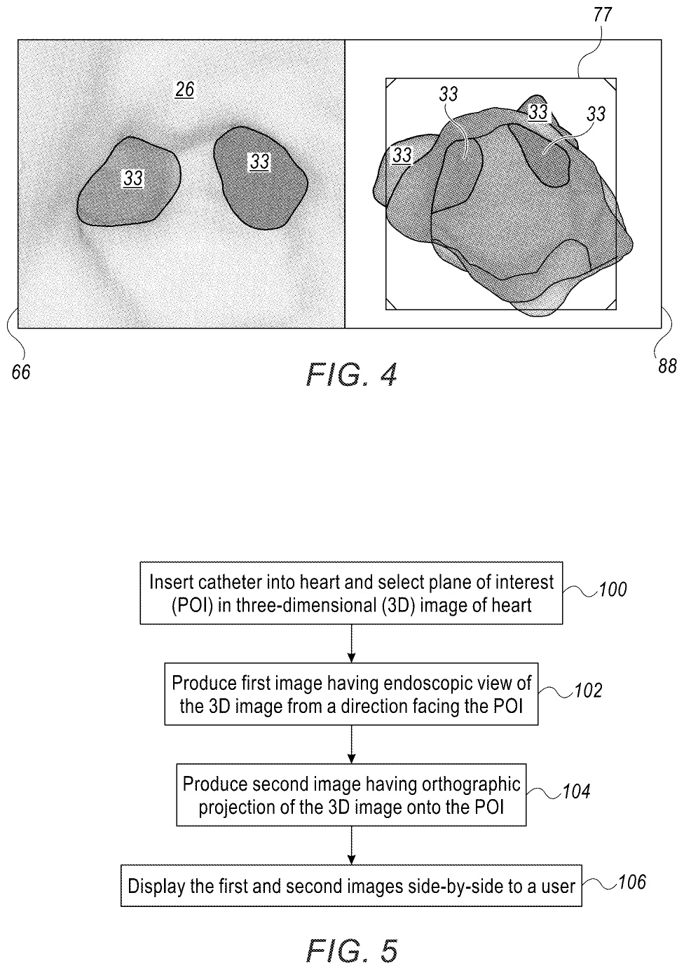

FIG. 4 is a schematic, pictorial illustration of a 3D visualization of heart 26 tissue and pulmonary veins (PVs) 33 using a side-by-side presentation of (i) sectional view 88 of heart 3D image clipped by POI 77, and (ii) endoscopic view 66 from a direction facing POI 77, in accordance with an example of the present disclosure.

In some examples, processor 42 is configured to present (e.g., to physician 30): (i) endoscopic view 66 produced using virtual camera 55, as described in FIG. 2 above, and (ii) sectional view 88 of the 3D image of heart 26 clipped by POI 77, which is shown in FIG. 3C above and whose production is described in detail in FIGS. 3A-3C above. In the present example, processor 42 is configured to display endoscopic view 66 and sectional view 88 side-by-side on display 46, but in other embodiments, processor 42 may display these images using any other suitable displaying configuration, as will be described in FIG. 5 below.

In some examples, during the tissue ablation procedure, physician 30 controls virtual camera 55 to produce the desired endoscopic view 66, and subsequently, controls processor 42 to produce sectional view 88 of the 3D image of heart 26, which is clipped by POI 77. Note that the sectional view of the 3D image clipped by POI 77 is not affected by the topography of PVs 33 and/or the section of heart 26. However, sectional view 88 does present a graphical representation of the topography of heart 26 and PVs 33. In other words, sectional view 88 ignores the topography, and therefore, provides the physician with a complementary view of the section of interest (and PVs 33). On the one hand, as described in FIG. 2 above, endoscopic view 66 provides physician 30 with a high-resolution image of PVs 33, but may have distortions in the displayed size of the heart elements, and in the distance therebetween. On the other hand, sectional view 88 displays, from the same gaze (e.g., viewing angle), a proportional size of the elements of heart 26, and of the distance between the heart elements.

In some examples, physician 30 may use sectional view 88 for estimating the real size of PVs 33 and the real distance therebetween. Based on this estimation, physician 30 and/or processor 42 may select, in endoscopic view 66, the sites for applying the RF ablation signals to the tissue of heart 26. Moreover, when physician 30 marks a selected ablation site on endoscopic view 66, processor 42 is configured to present a mark of the same ablation site on sectional view 88.

In other examples, physician 30 and/or processor 42 may mark the ablation sites on sectional view 88, and processor 42 may present the same ablation site over endoscopic view 66.

In both examples, physician 30 can see marks of one or more selected ablation sites displayed, at the same time, over: (i) the high-resolution image of endoscopic view 66, and (ii) the proportional image of sectional view 88. This side-by-side presentation helps physician 30 to determine the ablation sites accurately and conveniently, and therefore, to improve the quality of the ablation procedure.

FIG. 5 is a flow chart that schematically illustrates a method for displaying sectional view 88 of the 3D image of heart 26 clipped by POI 77, and endoscopic view 66 from a direction facing POI 77, in accordance with an example of the present disclosure.

The method begins at a POI selection step 100, with physician inserting DEA 40 into a cavity in question of heart 26, and selecting: (i) the position of virtual camera 55 within the 3D image of heart 26, and (ii) imaging parameters (e.g., direction and magnification) of virtual camera 55 for viewing a section of interest in heart 26.

In some examples, based on the illumination direction and imaging parameters of virtual camera 55, processor 42 is configured to select POI 77 within the 3D image of heart 26.

At a first image production step 102, processor 42 is configured to produce a first image, i.e., endoscopic view 66, based on the selected position and imaging parameters of virtual camera 55, Note that endoscopic view 66 is produced from a direction facing POI 77, as described in detail in FIGS. 2 and 3A above.

At a second image production step 104, processor 42 is configured to produce a second image. In the present example, the second image comprises sectional view 88 of the 3D image of the selected section of heart 26, clipped by POI 77, as described in detail in FIGS. 3A-3C above. Note that POI 77 is common to the first and second images, and therefore, both the first and second images are presenting the same section of heart 26 from the same direction, as described in detail in FIG. 4 above.

At a displaying step 106 that concludes the method, processor 42 is configured to display the first and second images to physician 30 and/or to any other user of system 20. In some examples, processor 42 is configured to display (on display 46) endoscopic view 66 and sectional view 88 side-by-side, as shown in FIG. 4 above. Moreover, processor 42 is configured to display, on one or both of endoscopic view 66 and sectional view 88, one or more marks of selected respective sites intended to be ablated using DEA 40, as described in FIG. 4 above.

In other examples, processor 42 is configured to toggle between the display of endoscopic view 66 and sectional view 88 on display 46. For example, processor 42 is configured to: (i) display endoscopic view 66 when applying to display 46 a first range of zoom values, and (ii) display sectional view 88 when applying to display 46 a second range of zoom values, different from the first range of zoom values.

In alternative embodiments, processor 42 is configured to display the 3D image of heart 26 (e.g., exterior view 54 of FIG. 2 above, or the image shown in FIG. 3A above) instead of endoscopic view 66 or sectional view 88, when applying to display 46 a third range of zoom values, different from the first range of zoom values and the second range of zoom values.

The method of FIG. 5 is simplified for the sake of conceptual clarity, and in other examples, processor 42 is configured to display two or more images of a selected section of heart 26 using any suitable displaying configuration. Moreover, this particular technique of graphical user interface (GUI) is shown by way of example, in order to illustrate certain problems that are addressed by examples of the present disclosure, and to demonstrate the application of these examples in enhancing the performance of such a mapping and ablation system. Examples of the present disclosure, however, are by no means limited to this specific sort of example system and/or medical application, and the principles described herein may similarly be applied to other sorts of medical systems used for performing any suitable sort of medical procedures that require a combination of high-resolution imaging and proportional images of the tissue in question, and presentation thereof using a suitable configuration of GUI.

The examples described herein mainly address producing multiple types of imaging, presentation of selected sections in a patient heart, and selection of sites for ablation tissue in the selected sections. The methods and systems described herein can also be used in other applications, such as in any system utilizing an endoscopic view. For example, in ear-nose-throat (ENT) applications using endoscopic views for navigating an ENT tool to a sinus of the ENT system of a patient.

### Example 1

A method including:
(i) inserting a catheter (22) into an organ (26) of a patient (28) and selecting, in a three-dimensional (3D) image (54) of the organ (26), a plane of interest (POI) (77);
(ii) producing a first image including an endoscopic view (66) of the 3D image (54) from a direction facing the POI (77);
(iii) producing a second image including a sectional view (88) of the 3D image (54) clipped by the POI (77); and
(iv) displaying the first and second images to a user (30).

### Example 2

The method according to Example 1, wherein producing the second image includes producing a graphic representation of a clip plane of the POI, and displaying the sectional view of the clip plane.

### Example 3

The method according to Example 1, wherein producing the first image includes positioning, within the 3D image of the organ, a virtual camera at a given position and a given orientation, and defining one or more imaging parameters for producing the endoscopic view.

### Example 4

The method according to Example 3, wherein the organ includes a heart and the 3D image includes a 3D image of at least a section of the heart, wherein positioning the virtual camera includes selecting the given position and the given orientation of the virtual camera for displaying a section of the heart, and wherein defining the one or more imaging parameters in the virtual camera, includes defining one or both of: (i) a magnification, and (ii) one or more angles of view, for producing the endoscopic view.

### Example 5

The method according to Example 3, wherein the section includes one or more pulmonary veins (PVs), and wherein the first and second images are used for performing a PV isolation procedure in at least one of the PVs.

### Example 6

The method according to Examples 1 through 5, wherein displaying the first and second images includes displaying the first and second images side by side.

### Example 7

The method according to Examples 1 through 5, wherein displaying the first and second images includes toggling between the display of the first and second images.

### Example 8

The method according to Example 7, wherein toggling between the display includes displaying the first image when applying to the display a first range of zoom values, and displaying the second image when applying to the display a second range of zoom values, different from the first range of zoom values.

### Example 9

The method according to Example 8, wherein the method includes displaying the 3D image instead of the first image or the second image, when applying to the display a third range of zoom values, different from the first range of zoom values and the second range of zoom values.

### Example 10

A system (22), including:
(i) a processor (42), which is configured to: (i) receive a selection of a plane of interest (POI) (77) in a three-dimensional (3D) image (54) of an organ (26) of a patient (28), (ii) produce a first image including an endoscopic view (66) of the 3D image (54) from a direction facing the POI (77), and (iii) produce a second image including a sectional view (88) of the 3D image (54) clipped by the POI (77); and
(ii) a display (46), which is configured to display the first and second images to a user (30).

### Example 11

The system according to Example 10, wherein the processor is configured to produce the second image by: (i) producing a graphic representation of a clip plane of the POI, and (ii) displaying on the display the sectional view of the clip plane.

### Example 12

The system according to Example 10, wherein the processor is configured to produce the first image by: (i) positioning, within the 3D image of the organ, a virtual camera at a given position and a given orientation, and (ii) defining one or more imaging parameters for producing the endoscopic view.

### Example 13

The system according to Example 10, wherein the organ includes a heart and the 3D image includes a 3D image of at least a section of the heart, wherein the processor is configured to select the given position and the given orientation of the virtual camera for displaying a section of the heart, and wherein the processor is configured to define in the virtual camera one or both of: (i) a magnification, and (ii) one or more angles of view, for producing the endoscopic view.

### Example 14

The system according to Example 13, wherein the section includes one or more pulmonary veins (P-Vs), and wherein the first and second images are used for performing a PV isolation procedure in at least one of the PVs.

### Example 15

The system according to Examples 10 through 14, wherein the processor is configured to display the first and second images on the display, side by side.

### Example 16

The system according to Examples 10 through 14, wherein the processor is configured to display the first and second images on the display, by toggling between the display of the first and second images.

### Example 17

The system according to Example 16, wherein the processor is configured to: (i) display the first image when applying to the display a first range of zoom values, and (ii) display the second image when applying to the display a second range of zoom values, different from the first range of zoom values.

### Example 18

The system according to Example 16, wherein the processor is configured to display the 3D image instead of the first image or the second image, when applying to the display a third range of zoom values, different from the first range of zoom values and the second range of zoom values.

It will thus be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A method, comprising:
inserting a catheter into an organ of a patient and selecting, in a three-dimensional (3D) image of the organ, a plane of interest (POI);
producing a first image comprising an endoscopic view of the 3D image from a direction facing the POI;
producing a second image comprising a sectional view of the 3D image clipped by the POI; and
displaying the first and second images to a user.

2. The method according to claim 1, wherein producing the second image comprises producing a graphic representation of a clip plane of the POI, and displaying the sectional view of the clip plane.

3. The method according to claim 1, wherein producing the first image comprises positioning, within the 3D image of the organ, a virtual camera at a given position and a given orientation, and defining one or more imaging parameters for producing the endoscopic view.

4. The method according to claim 3, wherein the organ comprises a heart and the 3D image comprises a 3D image of at least a section of the heart, wherein positioning the virtual camera comprises selecting the given position and the given orientation of the virtual camera for displaying a section of the heart, and wherein defining the one or more imaging parameters in the virtual camera, comprises defining one or both of: (i) a magnification, and (ii) one or more angles of view, for producing the endoscopic view.

5. The method according to claim 3, wherein the section comprises one or more pulmonary veins (P-Vs), and wherein the first and second images are used for performing a PV isolation procedure in at least one of the PVs.

6. The method according to claim 1, wherein displaying the first and second images comprises displaying the first and second images side by side.

7. The method according to claim 1, wherein displaying the first and second images comprises toggling between the display of the first and second images.

8. The method according to claim 7, wherein toggling between the display comprises displaying the first image when applying to the display a first range of zoom values, and displaying the second image when applying to the display a second range of zoom values, different from the first range of zoom values.

9. The method according to claim 8 and comprising displaying the 3D image instead of the first image or the second image, when applying to the display a third range of zoom values, different from the first range of zoom values and the second range of zoom values.

10. A system, comprising:
a processor, which is configured to: (i) receive a selection of a plane of interest (POI) in a three-dimensional (3D) image of an organ of a patient, (ii) produce a first image comprising an endoscopic view of the 3D image from a direction facing the POI, and (iii) produce a second image comprising a sectional view of the 3D image clipped by the POI; and
a display, which is configured to display the first and second images to a user.

11. The system according to claim 10, wherein the processor is configured to produce the second image by: (i) producing a graphic representation of a clip plane of the POI, and (ii) displaying on the display the sectional view of the clip plane.

12. The system according to claim 10, wherein the processor is configured to produce the first image by: (i) positioning, within the 3D image of the organ, a virtual camera at a given position and a given orientation, and (ii) defining one or more imaging parameters for producing the endoscopic view.

13. The system according to claim 10, wherein the organ comprises a heart and the 3D image comprises a 3D image of at least a section of the heart, wherein the processor is configured to select the given position and the given orientation of the virtual camera for displaying a section of the heart, and wherein the processor is configured to define in the virtual camera one or both of: (i) a magnification, and (ii) one or more angles of view, for producing the endoscopic view.

14. The system according to claim 13, wherein the section comprises one or more pulmonary veins (P-Vs), and wherein the first and second images are used for performing a PV isolation procedure in at least one of the PVs.

15. The system according to claim 10, wherein the processor is configured to display the first and second images on the display, side by side.

16. The system according to claim 10, wherein the processor is configured to display the first and second images on the display, by toggling between the display of the first and second images.

17. The system according to claim 16, wherein the processor is configured to: (i) display the first image when applying to the display a first range of zoom values, and (ii) display the second image when applying to the display a second range of zoom values, different from the first range of zoom values.

18. The system according to claim 16, wherein the processor is configured to display the 3D image instead of the first image or the second image, when applying to the display a third range of zoom values, different from the first range of zoom values and the second range of zoom values.
